(19) United States
(12) Patent Application Publication
(10) Pub. No.: US 2010/0041093 A1
(43) Pub. Date: Feb. 18, 2010

(54) DEVICES AND METHOD FOR ELECTROPHYSICAL CELL ANALYSES

(75) Inventor: Steffen Hering, Wien (AT)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: UNIVERSITAET WIEN, Wien (AT)

(21) Appl. No.: 12/513,683

(22) PCT Filed: Oct. 31, 2007
(86) PCT No.: PCT/EP2007/009467

§ 371 Date: Sep. 1, 2009

(30) Foreign Application Priority Data
Nov. 6, 2006 (DE) 10 2006 052 231.1

(51) Int. Cl. C12Q 1/02; C12M 1/00; C12M 1/34

(52) U.S. Cl. 435/29; 435/307.1; 435/288.1

(57) ABSTRACT

Disclosed is a liquid holder mechanism (10) which is designed to form a liquid environment at a capillary tip (22) of a patch clamp pipette (20) and includes a holding element (11.2) for retaining a liquid drop (1) and at least one rod (12.2, 12.3) for positioning the holding element (11.2) on the capillary tip (22). The at least one rod (12.2, 12.3) is designed such that the holding element (11.2) can be separated from the capillary (21) in a state in which the liquid holder mechanism (10) is connected to the patch clamp pipette (20). Also disclosed are a measuring instrument that is equipped with said liquid holder mechanism (10) as well as a method for electrophysically analyzing a biological cell with the help of the liquid holder mechanism (10).

The invention relates to a liquid holder device for electrophysiological analyses of biological cells, in particular a liquid holder device with the features of the preamble of claim 1. The invention furthermore relates to an electrophysiological measuring instrument that is equipped with such a liquid holder device and in particular comprises a patch-clamp pipette. The invention also relates to a method for the electrophysiological analysis of a biological cell using such a liquid holder device.

The detection of currents of ions through ion channels in the cell membrane of isolated biological cells by electrophysiological measurements is known. These measurements are also designated as patch-clamp measuring and the measuring instrument used for this purpose is designated as patch-clamp device. The patch-clamp device contains a capillary-shaped pipette (patch-clamp pipette) on whose tip a cell can be sucked up and measured. The patch-clamp pipette contains a measuring electrode for bleeding-off an electrophysiological potential from the cell or for measuring transmembrane ionic currents, respectively.

The transport properties of the ion channels are of interest in the basic research for the characterization of cytobiological processes and in particular in the pharmacology for the characterization of active substances. Thus, the ion channels represent important targets for drugs. A plurality of drugs acts selectively on certain ion channels so that a tool for pharmacological analyses is available with the patch-clamp technique.

It was previously customary in the practice to carry out patch-clamp methods manually, in which an experimenter operates a micromanipulator in order to take up a cell with the tip of the patch-clamp pipette and to carry out the electrophysiological measurement on this cell. The manual performing of patch-clamp methods is disadvantageous since the course of the method is expensive and places high demands on the manual capabilities of the experimenter. Furthermore, the number of the active substances to be analyzed and of the ion channels constantly rises, so that high throughput analyses can be realized manually only to a limited extent or not al all. It is, e.g., known that the human genome codes for approximately 400 ion channels whose transport properties are influenced by more than 100 channel proteins. When a specific interaction of a group of active substances with certain channel proteins is to be analyzed, the number of the combinations to be analyzed readily increases over a limit above which manual methods can no longer be used.

A further disadvantage of the manual methods is apparent in special tasks in which the action of different drugs on a certain cell is to be analyzed. Prior to the electrophysiological measurement the cell must be perfused with a solution of the particular drug analyzed. A perfusion device can be used here that is described, e.g., in DE 43 05 405 C1. However, due to the perfusion with different drug solutions, the conventional manual methods become even more complicated and expensive.

Attempts are known for developing automated patch-clamp methods that have proven, however, to have only limited applicability in the practice on account of a high errors rate and a low reproducibility of the electrophysiological measuring. A limitation exists in particular in the special analysis task of allowing different drugs to act on a single cell and of analyzing the effect.

It is known from US 2005/0241940 A1 to transfer, for the perfusion, the tip of the patch-clamp pipette with a cell set on it from a first drug solution into a second drug solution, a liquid environment being locally maintained on the pipette tip during the transfer. A spiral-shaped or tubular drop holder is used for this purpose that can be axially displaced with a rod running parallel to the longitudinal direction of the pipette tip from a retracted position in which the pipette tip with the cell is freely exposed into an advanced position in which the pipette tip is surrounded by the tubular drop holder. The desired liquid environment of the capillary tip can be formed with a liquid drop that is trapped in the drop holder by capillary forces.

A disadvantage of the conventional drop holder in accordance with US 2005/0241940 A1 consists in the coaxial relative movement of pipette tip and drop holder, which have a slight distance (capillary gap) in all relative positions. In order to avoid damage to the pipette or the cell, a complex precision drive is therefore required. A further disadvantage consists in the hindering of the perfusion of the cell in the changed drug solution, which is at first screened on all sides from the cell by the drop holder. This results in a relatively high expenditure of time for the perfusion that is disadvantageous for the reproducibility and comparability of a plurality of patch-clamp measurements on a cell. It can also be disadvantageous if the stability of the drop is insufficient, so that the drop can come loose from the tip by a rapid movement of the patch-clamp pipette and the cell is damaged by contact with the air. Finally, patch-clamp pipettes that are suitable for the technique described in US 2005/0241940 A1 can be difficult to produce and assemble in an automated manner on account of the placing of the drop holder.

The present invention is based on the objective of providing an improved liquid holder device with which disadvantages of conventional patch-clamp devices and in particular of conventional drop holders are overcome. The invention is furthermore based on the objective of providing an improved measuring instrument, in particular for patch-clamp analyses. Finally, the invention is also based on the objective of providing an improved method available for the electrophysiological analysis of biological cells with which the disadvantages of the conventional patch-clamp methods are reduced or overcome.

These objectives are solved by a liquid holder device, a measuring instrument and an analysis method with the features of the independent claims. Advantageous embodiments and uses of the invention can be found in the dependent claims.

According to a first aspect the invention is based on the general technical teaching of providing a generic liquid holder device, adapted to form a liquid environment on a capillary tip of a patch-clamp pipette, with a holding element for taking up a liquid drop which holding element is arranged in such a manner that it can be separated from the capillary when used to hold liquid with a patch-clamp pipette. The at least one rod for holding the holding element is designed in such a manner that the holding element can be separated, especially from the capillary tip.

It is provided in particular that the holding element can be positioned with the rod on or adjacent to the capillary tip (conditioning position) and can be separated from the capillary so far (release position) that the capillary tip is free and that a distance between the holding element and the capillary tip is greater than the width of a capillary gap. In contrast to the conventional drop holder the holding element—capillary tip distance when the holding element is separated from the capillary tip, i.e., in the release position is in particular greater than in the conditioning position. A number of advantages in relation to the requirements as to the precision of the movement of the holding element as well as in relation to the intended use of the patch-clamp pipette can advantageously be achieved by the enlarging of the holding element—capillary tip distance in the release position.

According to a first embodiment of the invention the rod on which the holding element is fastened is arranged in such a manner that it can be pivotably fixed on a capillary holder of the patch-clamp pipette. The rod preferably comprises a pivot hinge on an end opposite the holding element. The holding element has a free surface on at least one side that can be approached to the capillary tip from the side, i.e., radially in reference to a longitudinal direction of the capillary. The inventor determined that it is sufficient for a reliable maintaining of a liquid environment on the tip of the capillary if the drop is held by the holding element, which generally comprises a curved component part with a lateral opening or comprises a level component part. Simultaneously, the laterally exposed surface, in particular the opening provided on the side of the holding element, makes possible a simplified course of the movement of the rod with the holding element.

According to the first embodiment the rod is pivotable relative to the longitudinal direction of the capillary of a patch-clamp pipette when the liquid holder device is attached to the patch-clamp pipette. The holding element can be pivoted by the pivoting of the rod from a closed state (conditioning position), in which the holding element is separated from the capillary tip by a capillary gap or rests on the capillary tip, by a radial movement into a projecting state (release position), in which the holding element releases the capillary tip, e.g., for further observations, measurements or cell manipulations. The axial movement of the conventional drop holder is advantageously replaced by a radial movement of the holding element in accordance with the invention.

According to a second embodiment of the invention the holding element for holding the liquid drop is arranged for the formation of the liquid environment on the capillary tip under the action of gravitational force, the holding element being detachably fastened on the rod. The holding element advantageously forms a support on which an amount of liquid is carried. In this instance, in distinction to the conventional drop holder larger amounts of liquid can be provided for conditioning the capillary tip with improved stability and reliability.

In the first as well as in the second embodiment of the invention the holding element can have a form that is open on one side, level or curved. This results as a further important advantage in an improved perfusion during the transfer of a liquid-encased cell on the tip of the capillary into a changed surrounding medium. The cell is only partially screened by the holding element from the new surrounding medium so that the drop on the drop holder is rapidly diluted by a liquid flow and diffusion. As a result, the effect of the substance in the new surrounding medium, e.g., a new drug solution can be analyzed after a time that is shortened in the comparison to the time necessary for the perfusion with a conventional drop holder.

A further advantage of the liquid holder device of the first embodiment of the invention is that no special requirements must be placed on the form of the holding element. In general, the holding element can be a level or curved component part that is arranged adjacent to the capillary tip in the closed state of the liquid holder device or partially surrounds it azimuthally. The holding element is areal, e.g., built in the form of a glass or plastic lamella.

The holding element is arranged to receive (hold) a liquid drop. In the first embodiment of the invention, the liquid drop is trapped in a stable manner under the action of capillary forces. It can be provided that the drop is held by the capillary forces jointly exerted in the closed state of the liquid holder device by the tip of the capillary and the holding element. However, an embodiment of the invention is preferred in which the holding element is arranged to hold a liquid drop alone without further fixed boundary surfaces under the action of capillary forces. In this instance, a liquid drop can be reliably held on the holding element in a projecting state of the liquid holder device, which avoids the settling of gas bubbles on the holding element.

If the holding element comprises an areal component part in accordance with a preferred variant of the first embodiment of the invention, a relatively large amount of liquid can advantageously be obtained in the environment of the tip of the capillary. This reduces the danger of an unintended drying out of the cell during the transfer into a changed surrounding medium. A variant is especially preferred in which the holding element is formed by a section of a hollow line cut in axial direction. The wall of the hollow line forms the holding element open on one side. If the holding element has the form of a hollow cylinder cut in axial direction, advantages can result for the formation of a uniform liquid environment on the capillary tip. The hollow cylinder can have, e.g., a circular or elliptical base surface. In this case the drop holder is arranged so that it surrounds the pipette tip in such a manner in the closed state of the liquid holder device that this tip is arranged in the center or focal point of the circular or elliptical base surface.

Alternatively, a level or a profiled plate can be provided as holding element. The profiled plate has, e.g., a form with several level partial surfaces that are bent toward the tip of the capillary, or with a profile adapted to the form of the capillary tip of the capillary.

If the holding element is provided with an electrode in the first or second embodiment of the invention the structure of the liquid holder device can be simplified. The electrode can be used as reference electrode for patch-clamp measurements. The electrode on the holding element or a line connected to the electrode is preferably connected to the rod of the drop holder. The rod especially preferably forms an integrated component part with the holding element and the electrode (optionally with the line).

The liquid holder device in accordance with the invention can advantageously be readily used in automated methods. In the first embodiment of the invention positioning movements for transferring the drop holder from the closed state to the projecting state can be carried out without problems with a positioning mechanism provided on a patch-clamp device. However, a variant of the first embodiment of the invention in which the liquid holder device is provided with an independent drive device is preferred. The drive device is arranged, e.g., for the piezoelectric pivoting of the rod of the liquid holder device and provided for achieving a large positioning path, preferably on the pivot hinge.

An important advantage of the invention is the compatibility of the liquid holder device according to the invention with different capillary forms and in particular pipette forms. The pivot hinge of the liquid holder device is preferably arranged for the detachable connection with the capillary. In a corresponding manner, existing patch-claim pipettes can be retrofitted with the liquid holder device. A clamping of the liquid holder device on the patch-clamp pipette, in particular a capillary holder of the patch-clamp pipette, via a clip connection (bracket connection) provided on the pivot hinge is provided in an especially preferred manner. However, it is not obligatorily necessary that the liquid holder device is arranged for the detachable connection with the capillary, e.g., the patch-clamp pipette. Alternatively, a fixed connection can be provided.

In the second embodiment of the invention the rod of the liquid holder device is preferably arranged for the rigid fastening to the capillary or the capillary holder of the patch-clamp pipette so that the structure of the liquid holder device and of the patch-clamp pipette is advantageously simplified. According to a preferred variant the holding element has the form of a cup in whose wall an insertion opening for receiving an end of the rod is provided. If the insertion opening and the interior of the cup are connected according to a further preferred variant of the second embodiment of the invention via a contact groove, advantages result for the measuring with a reference electrode formed by the rod or connected to the rod.

The holding element of the liquid holder device in accordance with the invention is arranged corresponding to the preferred applications of the invention in cytobiological analyses for taking up a slight amount of liquid (volume in particular 0.05-100 μl), e.g., in the form of a drop, of a liquid comprising water, an aqueous saline solution such as, e.g., a physiological solution, an aqueous solution or suspension with biological macromolecules or with cells or an aqueous-oily emulsion. The trapping action of the holding element can advantageously be improved more in both embodiments of the invention if the holding element carries a hydrophilic or an oleophilic inner coating. The inner coating is formed by hydrophilic or oleophilic substances such as are known, e.g., from functionalized substrates in the cell biology.

An electrophysiological measuring instrument having a capillary with a capillary tip for receiving a biological cell and having a liquid holder device in accordance with the invention connected to the capillary constitutes an independent subject matter of the invention. The capillary is preferably part of a patch-clamp pipette or of a microelectrode device.

The patch-clamp pipette preferably includes a capillary holder to which the capillary can be fixed detachably. The capillary holder can advantageously be used as a gripper for further manipulations, e.g., in the production of the capillary. It is especially preferred that the capillary is connected to the capillary holder via a conical plug connection.

If the measuring instrument is equipped with a drive device arranged for moving and positioning the patch-clamp pipette and the liquid holder device, advantages can be achieved for an automated operation of the measuring instrument. According to a further preferred variant of the invention the measuring instrument can be provided with a pipette drawing device arranged for the production of the capillary. The capillary can advantageously be produced therewith immediately before an electrophysiological measurement and be used free of damage or contaminations.

According to a further aspect of the invention the above-named objective is solved by the general technical teaching of carrying out a method for the electrophysiological analysis of a biological cell using the liquid holder device in accordance with the invention. Patch-clamp measurements can be advantageously carried out during the transfer of a cell into a changed surrounding medium and during a perfusion with the latter.

Further details and advantages of the invention will now be described making reference to the accompanying drawings, in which:

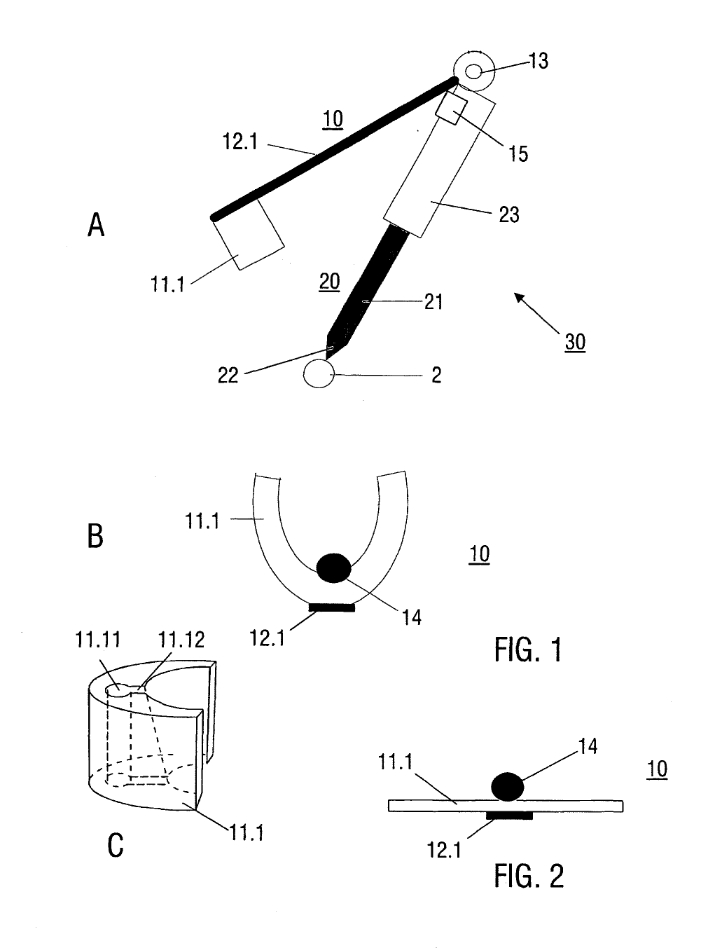

FIGS. 1 to 4: show variants of the first embodiment of the liquid holder device in accordance with the invention;

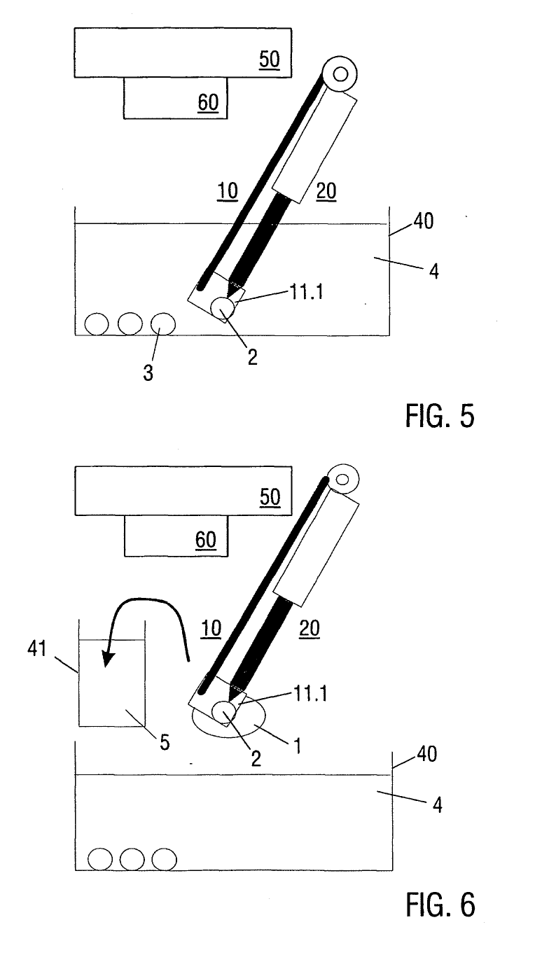

FIGS. 5 and 6: show a schematic illustration of the transfer in accordance with the invention of a cell into a changed surrounding medium using the liquid holder device in accordance with the first embodiment of the invention;

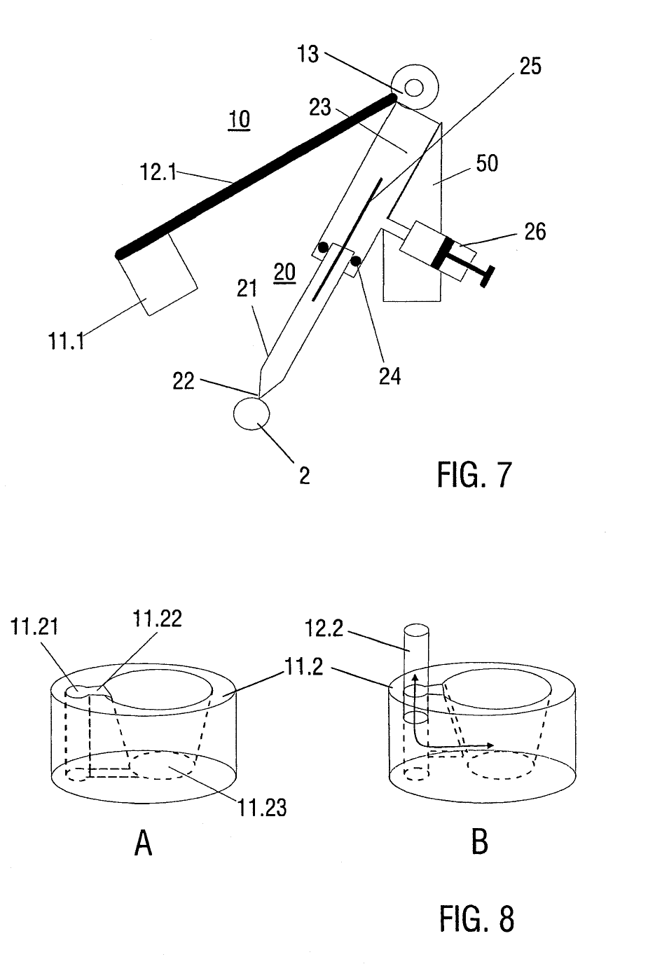

FIG. 7 shows a further variant of the first embodiment of the liquid holder device in accordance with the invention;

FIG. 8 shows a preferred variant of the holding element used in the second embodiment of the invention;

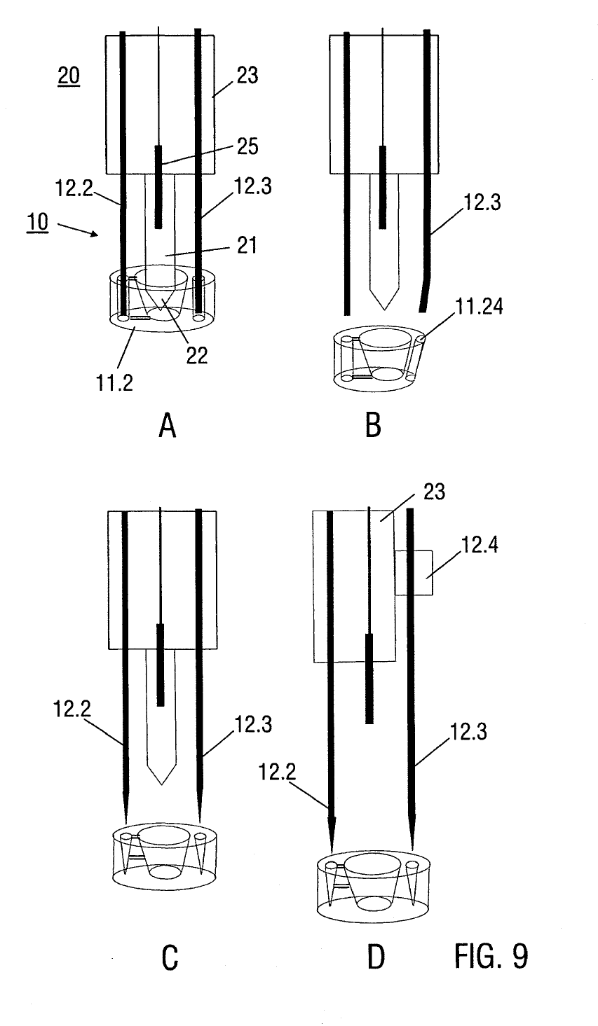

FIG. 9 shows variants of the second embodiment of the liquid holder device in accordance with the invention;

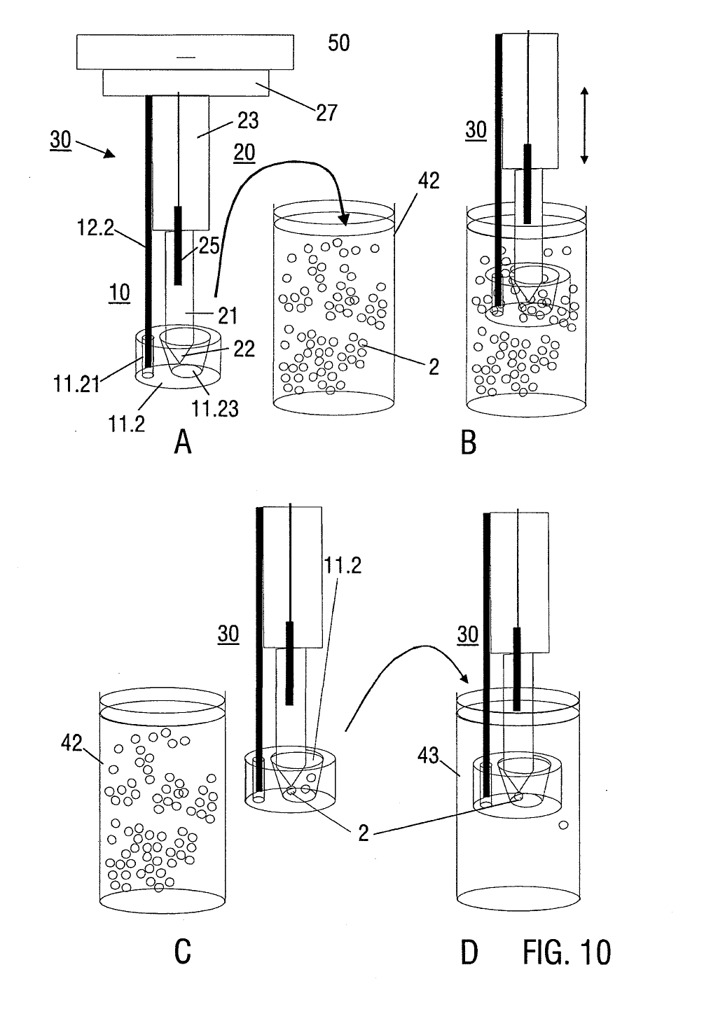

FIG. 10 shows a schematic illustration of the transfer in accordance with the invention of a cell into a changed surrounding medium using the liquid holder device in accordance with the second embodiment of the invention;

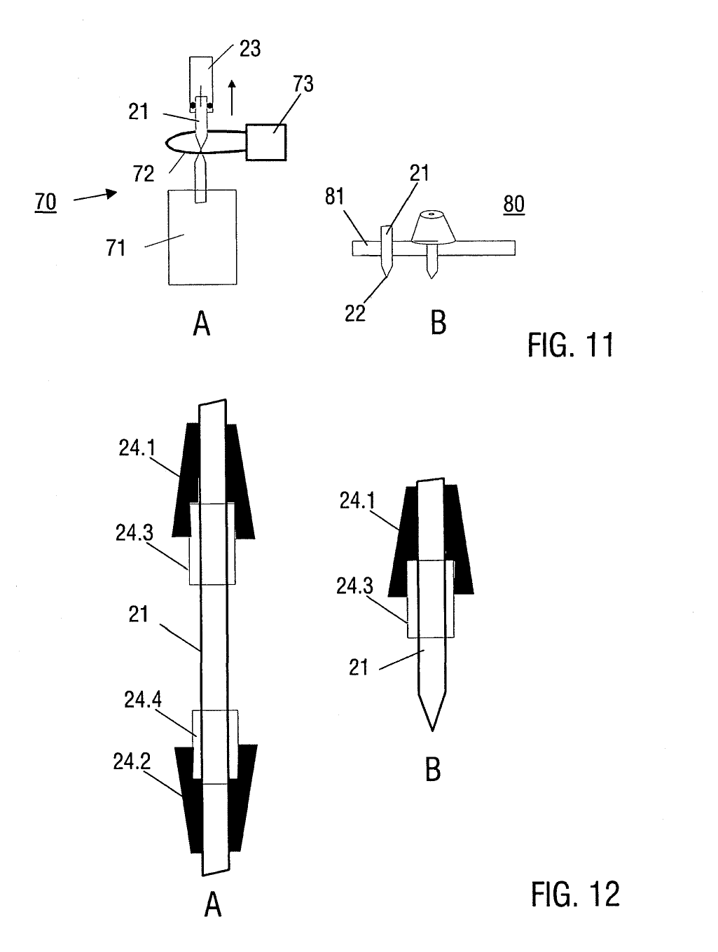

FIG. 11 shows illustrations of additional parts of a measuring instrument for electrophysiological analyses;

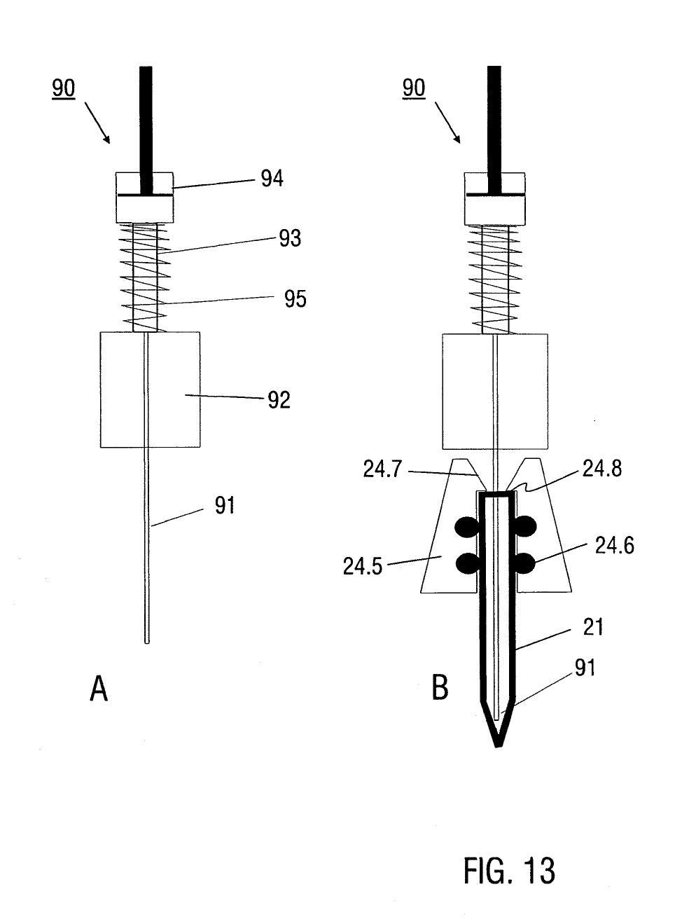

FIG. 12 shows a schematic illustration of a capillary holder of a patch-clamp pipette used with preference;

FIG. 13 shows illustrations of the filling of the capillary of a patch-clamp pipette with a filling device; and

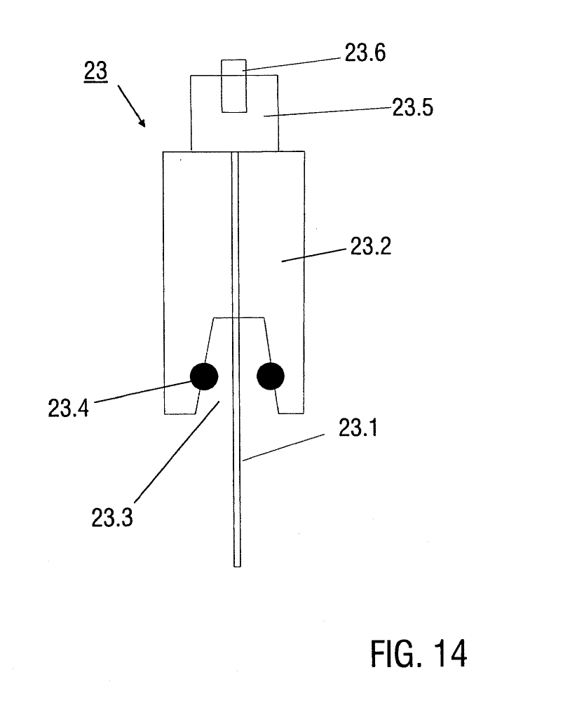

FIG. 14 shows a schematic illustration of a capillary holder of a patch-clamp pipette.

The invention is described in the following with exemplary reference made to a preferred embodiment of the liquid holder device, which is attached to a pipette tip of a patch-clamp device. Details of the patch-clamp technique are not described here since they are generally known from the state of the art (see, e.g.: O. P. Hamill et al. in “Pflugers Arch.”, vol. 391(2), 1981, pp. 85-100). The liquid holder device can be used not only with a patch-clamp pipette but rather can also be combined with other measuring instruments for electrophysiological analysis such as, e.g., with microelectrode devices, ion-selective microelectrodes, etc.

FIG. 1A shows a liquid holder device 10 with the holding element 11.1, the rod 12.1 and the pivot hinge 13 in accordance with a variant of the first embodiment of the invention. The liquid holder device 10 is shown in combination with a patch-clamp pipette 20 in the projecting state of the holding element 11.1. The holding element 11.1 is separated in this state from the capillary tip of the patch-clamp pipette 20. FIG. 1B shows an enlarged sectional view of the holding element 11.1, which is arranged to take up a liquid drop (see FIG. 4). The patch-clamp pipette 20 is constructed in a known manner with a capillary 21 that has in particular a free capillary tip 22 for sucking up a biological cell 2 and an integrated measuring electrode (not shown) and is fastened to a capillary holder 23. The capillary tip 22 typically has a tip diameter in the μm range. The capillary holder 23 is constructed, for example, as is illustrated in FIG. 14.

The compound consisting of the liquid holder device 10 and the patch-clamp pipette 20 forms a measuring instrument 30 in accordance with the invention (partially shown) that furthermore comprises line connections for connection to a control device and is optionally equipped with a drive device (see FIG. 7).

The holding element 11.1 has the form of an axially cut hollow cylinder with an elliptical base surface. A semi-tubular chamber (so-called semi-tube chamber) is formed by the holding element 11.1, which chamber is arranged to receive the capillary 21 of the patch-clamp pipette 20 and a liquid drop. The electrode 14 is arranged on the inner surface of the holding element 11.1. On the outside the holding element 11.1 is connected to the rod 12.1. The electrode 14 extends along the length of the rod 12.1.

The holding element 11.1 consists of glass or plastic (e.g., PVC) with a wall thickness of around 200 μm to 1 mm and a length in the longitudinal direction of the rod 12.1 of around 3 mm to 10 mm. The lateral opening of the holding element 11.1 has a width of around 1.5 mm to 3 mm.

The electrode 14 is formed by a metallic layer or metal wire on the side (in particular the inner side) of the holding element 11.1 which faces the capillary 21. The electrode 14 consists, e.g., of silver/silver chloride (Ag/AgCl) or another electrically conductive material, e.g., carbon or platinum. Furthermore, the inner side of the holding element 11.1 can be coated hydrophilically.

If a contact of the cell to be analyzed with a conductive material of the electrode is to be avoided (in case the cell could be influenced, e.g., by Ag/AgCl), one of the following measures is provided. Firstly, the electrode can consist of agar-agar and an Ag/AgCl electrode part, in which the agar-agar is connected via a salt bridge to the electrode part. Secondly, the electrode can be partially coated or arranged on the outside of the holding element 11.1 so that a diffusion path is formed, e.g., from Ag/AgCl to the cell.

The rod 12.1 serves to hold the holding element 11.1. The rod 12.1 is a straight, elongated component part with a longitudinal direction from which the holding element 11.1 projects radially. The holding element 11.1 opens in radial direction relative to the longitudinal direction of the rod 12.1. The rod 12.1 is connected on its first (proximal) end to the pivot hinge 13 whereas the holding element 11.1 is fastened on the second, free (distal) end of the rod 12.1. The rod 12.1 consists of an inert, electrically insulating material such as, e.g., plastic (e.g., PE) or of a ceramic material. The electrode 14 or a line connection connected to it is integrated into the rod 12.1 or fastened on its surface.

According to an alternative variant the electrode 14 and the rod 12.1 are not two different components but rather the rod forms the electrode. If the rod 12.1 and optionally the holding element 11.1 are arranged so as to form the electrode, they consist at least partially of a conductive material such as, e.g., of Ag/AgCl. When using agar-agar the electrode 14 (or, correspondingly, the rod 12.1) preferably comprises a small glass tube in which the jelly-like agar-agar is arranged, that then changes into a salt bridge.

The pivot hinge 13 comprises, e.g., a hinge or a thin-layer articulation that is connected on the one hand to the rod 12.1 and on the other hand to the patch-clamp pipette 20 or to an appropriate clamping mechanism.

FIG. 1A schematically illustrates a piezoelectric drive device 15 arranged for pivoting the rod 12.1 about an axis vertical to the longitudinal direction of the patch-clamp pipette 20. The drive device 15 is arranged, e.g., between a surface of the patch-clamp pipette 20 and the rod 12.1. When the drive device 15 is loaded with a positioning voltage, a piezoelectric element of the drive device 15 expands, so that the rod 12.1 can be pivoted.

FIG. 1C shows a further variant of a holding element 11.1 with a bent form, the holding element 11.1 being provided for the detachable connection on the rod 12 of the liquid holder device 10 (see FIG. 1A). To this end, the holding element 11.1 is formed, e.g., using the injection molding method with an insertion opening 11.11 and a semi-tubular chamber for receiving the capillary tip. The rod 12, which can form the reference electrode at the same time, is inserted into the insertion opening 11.11. Electrical contact with the chamber, which is filled with liquid in the state of the approach to the capillary tip, is produced by a contact groove 11.12.

The variant of the holding element 11.1 shown in FIG. 1C can be modified in such a manner that the chamber is formed in a tubular manner and that the capillary tip (not shown) can be completely surrounded. The holding element 11.1 has the form of a cup with open bottom. Due to the detachable connection with the rod 12 the holding element 11.1 can be separated from the capillary tip. The holding element 11.1 is dimensioned in such a manner that the fluid is held in the holding element 11.1 by the action of capillary forces.

Alternatively to the holding element 11.1 shown with a bent form, a holding element 11.1 in the form of a plane plate, e.g., of glass or plastic can be provided, according to FIG. 2, on the distal end of the rod 12.1. The plane holding element 11.1 carries the electrode 14 on the side facing the capillary. Given a suitable shape of the rod 12.1 the holding element 11.1 can be connected in one piece to the end of the rod 12.1.

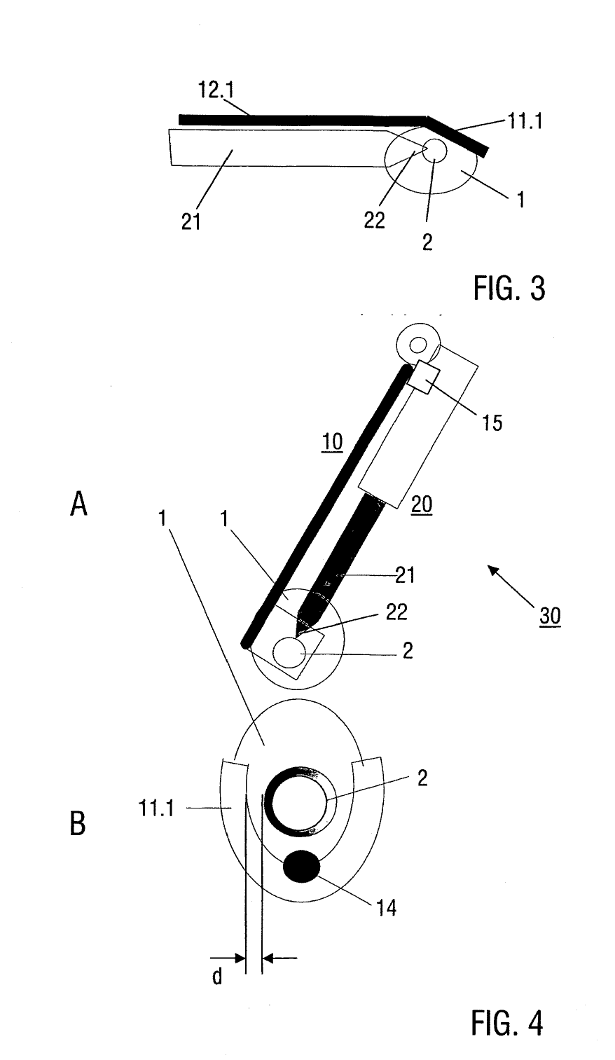

According to a further alternative the plane plate of the holding element 11.1 can be bent in the axial direction of the capillary 21 (FIG. 3). The holding element 11.1 is advantageously adapted in this case to the form of the capillary tip 22 of the capillary 21. As a result, when the danger of an unintended drying out is low, the liquid drop 1 on the holding element 11.1 can be formed smaller than in the variant in accordance with FIGS. 1 and 2 and the dead volume around the cell 2 can be reduced correspondingly. Alternatively to the form of the bent plate shown, a more complex form in accordance with the outer contour of the capillary tip 22 can be provided. Furthermore, FIG. 3 illustrates that the holding element 11.1 and with it the electrode can directly abut the capillary 21. It is alternatively possible that the rod 12.1 is arranged in such a manner with the drive device 15 (FIG. 1A) that the electrode is arranged in the direct vicinity of the capillary tip 22.

The FIGS. 4A and 4B show the first embodiment according to FIG. 1 corresponding to the closed state of the liquid holder device 10 with a liquid drop 1 and a cell 2. There is only a capillary gap between the capillary tip 22 in the inner surface of holding element 11.1, or the inner surface of holding element 11.1 contacts the capillary tip 22. The liquid drop 1 is held on the capillary 21 by the capillary forces even when the patch-clamp pipette 20 is withdrawn from a liquid surrounding medium.

The FIGS. 5 and 6 schematically illustrate the course of a method in accordance with the invention for the electrophysiological analysis of a biological cell using the first embodiment of the invention with further details.

Cells of a cell culture 3 are located on the bottom of a first culture container 40 filled with a first surrounding medium 4. The surrounding medium 4 comprises, e.g., a cultivation medium of the cell culture 3. In a first method step a cell 2 is removed in a known manner from the cell culture 3. The cell removal preferably takes place while the liquid holder device 10 is in the projecting state and the capillary 21 of the patch-clamp pipette 20 can move freely onto the cell culture 3, be manipulated and be observed with the schematically shown image recording device 60. The movement of the liquid holder device 10 and of the patch-clamp pipette 20 takes place with a schematically shown drive device 50.

A pivoting of the rod 12.1 subsequently follows for transferring the liquid holder device 10 into the closed state. This situation is illustrated in FIG. 5. The holding element 11.1 surrounds the tip 22 of the capillary with the cell 2. In this situation a first patch-clamp measurement can be carried out on the cell 2.

The transfer of the cell 2 into a further culture container 41 with a changed surrounding medium 5 subsequently takes place in accordance with FIG. 6. For this, the relative position between the patch-clamp pipette 20 and the culture containers 40, 41 is changed using the schematically illustrated drive device 50 and a control device (not shown) in that, e.g., the patch-clamp pipette 20 is moved is moved from the first culture container 40 to the second culture container 41.

Due to the action of the capillary forces a drop formation occurs on the holding element 11.1 during the lifting of the patch-clamp pipette 20 out of the first surrounding medium 4. The contact between the cell 2 and the electrode 14 is maintained, so that the measuring is not disturbed when the cell 2 is moved out of the first surrounding medium 4.

During the immersion of the patch-clamp pipette 20 into the second surrounding medium 5 the drop 1 on the tip 22 of the patch-clamp pipette 20 is rapidly diluted by the second surrounding medium 5. During the dilution the measuring of cell potentials or ionic currents through the cell membrane can advantageously be continued in order to directly detect the action of the second surrounding medium 5. The running measurement during a rapid exchange of the surrounding liquid of the cell 2 is especially advantageous during the measurement of ligand-activated ionic currents.

The steps of changing of the surrounding medium with running or a repeated measuring of the cell potentials in accordance with the FIGS. 5 and 6 can be repeated as a function of the concrete task posed. The number of repetitions is advantageously practically unlimited, so that the invention is especially suited for the analysis of a plurality of test media, in particular in the framework of high-throughput methods. The conventional application of test media via hoses can advantageously be dispensed with, which not only increases the accuracy of the measuring but also increases the possible number of the applicable test media and therewith the measuring throughput.

FIG. 7 shows a further variant of the first embodiment of the liquid holder device 10 in accordance with the invention with the components 11.1, 12.1 and 13 on a patch-clamp pipette 20. The holding element 11.1 is shown as in FIG. 1A in the projecting state. The liquid holder device 10 and the patch-clamp pipette 20 are connected to the drive device 50 that comprises, for example, a 3-axis manipulator. FIG. 7 shows the pivot hinge 13 by way of example on the upper end of the capillary holder 23. Alternatively, the pivot hinge 13 can be arranged laterally on the capillary holder 23.

The capillary 21 of the patch-clamp pipette 20 is detachably fastened on capillary holder 23. The capillary holder 23 has a suction device 26 for producing a vacuum under whose action the cell 2 can be held on the capillary tip 22. As shown, the capillary 21 is preferably placed in a seal 24, e.g., of rubber on the inner side of the capillary holder 23, with the measuring electrode 25, e.g., a silver wire, running through the capillary holder 23 to further line connections (not shown). The capillary holder 23 advantageously forms a gripper with the seal 24 that can be used when drawing the capillary 21 (see FIGS. 11, 12). Alternatively, a conical plug connection can be provided with which the capillary 21 can be more readily replaced (see FIG. 12). In this case capillaries 21 are used, which have a conical form on their back end.

In the second embodiment of the invention the holding element is arranged for providing the liquid for forming the liquid environment on the capillary tip under the action of the gravitational force in a container, e.g., a cup, into which the capillary tip extends in the assembled state of the liquid holder device. In this case the holding element can not be separated from the capillary tip by the above-described pivoting movement since the container edge would otherwise damage the capillary tip. In a corresponding manner, the second embodiment of the invention provides that the holding element is detachably fastened on the rod of the liquid holder device. A preferred variant of the holding element 11.2 used for the second embodiment of the invention is illustrated in the FIGS. 8A and 8B.

The holding element 11.2 according to FIG. 8A has the form of a cup in whose wall an insertion opening 11.21 is provided. The cup furthermore has a closed bottom 11.23 and a contact groove or a contact gap 11.22 through which an electrode arranged in the insertion opening 11.21 makes electrical contact with the inside of the cup via the liquid present in the cup and the contact groove 11.22.

The holding element 11.2 consists, e.g., of a plastic formed using the injection-molding method. For example, an elastomeric plastic can be provided. In this case the rod (not shown in FIG. 8A) can be tightly fitted in the insertion opening 11.21 under the action of an elastic tension. Alternatively or additionally, a snap or catch connection (not shown) can be provided on the insertion opening 11.21. The holding element 11.2 according to FIGS. 8A and 8B has, for example, the following dimensions: Outside diameter: 3 mm to 10 mm, cup diameter: 0.3 mm to 3 mm, and axial height 2 mm to 10 mm.

FIG. 8B illustrates the combination of the holding element 11.2 with a hollow rod 12.2 (partly shown) that can be advantageously used for the transport of liquid into or out of the cup. The rod 12.2 has the form of a tube that is open on its free end on the holding element 11.2 and is connected on the opposite end to a liquid reservoir. Solution can be supplied into the cup or removed from it by suction through the rod 12.2 via the contact gap.

The provision of the contact gap respectively of the contact groove has the particular advantage that in the tubular embodiment of the rod 12.2 the amount of liquid in the holding element can be changed by a change of pressure in the rod 12.2.

According to a modification the holding element can be formed as cup with an open bottom so that the variant of the first embodiment of the invention cited above with reference made to FIG. 1C results.

According to a further modification the holding element 11.2 can have two insertion openings for receiving two rods of the liquid holder device. Different variants of the holding element 11.2 with two insertion openings are illustrated in the FIGS. 9 and 10. The rods can be inserted into the insertion openings positively locking or force-fitting. The outside diameter of the rods and the inside diameter of the insertion openings are correspondingly adapted. FIG. 9 shows the inside diameters of the insertion openings on an enlarged scale by way of illustration.

The liquid holder device 10 shown in FIG. 9A comprises the holding element 11.2 and the rods 12.2, 12.3. The patch-clamp pipette 20 comprises the capillary 21 and the capillary holder 23. The measuring electrode 25, which is connected to a measuring head (not shown in FIG. 9), runs in the inside of the capillary 21. The rods 12.2, 12.3 of the liquid holder device 10 are firmly connected to the capillary holder 23 (e.g., adhered or screwed) in such a manner that they are symmetrically arranged on opposite sides of the capillary 21. The rods 12.2, 12.3 extend parallel to the axial extent of the capillary 21. The length of the rods 12.2, 12.3 is selected in such a manner that the capillary tip 22 extends into the cup of the placed-on holding element 11.2 without contacting the cup bottom. One of the rods 12.2, 12.3 can be used as electrode. Accordingly, a contact groove (contact gap) is provided on one of the insertion openings. The rods 12.2, 12.3, that can be tubular, form a detachable plug connection with the holding element 11.2.

FIG. 9B shows a modification in which one of the rods (12.3) has a curvature on the end that serves to fix the holding element 11.2. The rod 12.3 can be formed from a resilient material and engage into an oblique groove on the side of the holding element 11.2.

The FIGS. 9C and 9D show further modifications in which the free ends of the rods 12.2, 12.3 have tips that engage into conically formed insertion openings of the holding element 11.2. According to FIG. 9D a lateral screw coupling 12.4 of the rod 12.3 to the capillary holder 23 can be provided. Alternatively, the screw coupling 12.4 can be replaced by a ring-shaped holder. In this case, the advantage results that the rods can be readily put onto different types of capillary holders.

Referring to the FIGS. 10A to 10D, the course of a method in accordance with the invention for the electrophysiological analysis of a biological cell using the second embodiment of the invention with further details is described.

At first, the preparation of the patch-clamp pipette 20 takes place. Here, the production of the capillary 21 is preferably provided immediately before the electrophysiological analysis with a pipette drawing device described below with reference made to FIG. 11. The capillary 21 is received by the capillary holder 23. Subsequently, the patch-clamp pipette 20 is moved with the drive device 50 to a prepared holding element 11.2 in order to receive the latter by inserting the rod 12.2 into the insertion opening 11.21.

Subsequently, the measuring instrument 30 is moved with the drive device 50 to a suspension container 42. Suspended cells 2 are present in the suspension container 42. The patch-clamp pipette 20 is introduced with the drop holder device 10 into the cell suspension. The patch-clamp pipette 20 is aligned here in such a manner that the capillary 21 runs vertically (vertical to the bottom of the suspension container 42). When the capillary 21 is immersed with the holding element 11.2 into the cell suspension the latter is moved and thoroughly mixed. To this end the measuring instrument 30 is preferably moved up and down with the drive device 50 (FIG. 10B).

As a result, suspended cells are present in the cup of the holding element 11.2. Subsequently, the measuring instrument 30 is stopped. A vacuum is produced in the pipette of holder 23 and the capillary 21 with a suction device (see FIG. 7, not shown in FIG. 9), under the action of which a cell is sucked up at the capillary tip 22 after a short time. The so-called “gigaseal” connection necessary for the patch-clamp measuring is produced and the patch-clamp measuring known as such is started.

After a predetermined measuring time a change of liquid takes place in the environment of the measured cell 2. This measuring time is determined, for example, as a function of the value of the measured resistance over the cell (so-called “seal resistance”). For the change of liquid the measuring instrument 30 is lifted out of the first suspension container 42 (FIG. 10C), while the holding element 11.2 shown remains in the attached state and a liquid environment of the cell 2 is formed on the capillary tip 22. The measuring instrument 30 is transferred with the drive device 50 into the further suspension container 43 (FIG. 10D) in which a test solution is located. A rapid exchange of liquid takes place in the test solution with the liquid in the cup of the holding element 11.2. During the dilution of the surrounding medium of the cell 2 with the test solution the measuring of cell potentials can be continuously continued in order to directly detect the action of the test solution. As was mentioned above in connection with the first embodiment, the measuring of the cell potential can also be repeated in different test media when using the second embodiment.

When a tubular rod 12.2 or 12.3 is used, the amount of liquid in the cup can be changed, as a result of which the dilution time of the solution in the cup can be shortened by removing a volumetric portion by suction.

After the last measurement the deposition of the cell 2 in a cell culture, e.g., on the bottom of a culture container, can be provided. To this end the holding element 11.2 is removed from the capillary 21 (transfer into the release position). The removal of the holding element 11.2 from the rod 12.2 takes place, e.g., using a fork-shaped tool that is provided in the culture container or in the open air.

According to a preferred realization of the invention the measuring instrument 30 is provided with a pipette drawing device 70 schematically shown in FIG. 11A. Since capillaries for patch-clamp pipettes are very sensitive, become easily contaminated and can therefore hardly be transported, the pipette drawing device 70 can be advantageously used to produce the capillaries 21 of the patch-clamp pipette 20 at the site of the measuring instrument 30. In particular, an automated operation of the production and receiving of the patch-clamp pipette 20 is possible.

The pipette drawing device 70 comprises a clamping block 71, a heating wire 72 and a heating 73. In order to draw out a capillary 21, at first a glass capillary with a measuring electrode wire is inserted into the clamping block 71. Subsequently, the capillary holder 23 is set onto the free end of the glass capillary. For this, the drive device (50, see, e.g., FIGS. 5, 6 10) is preferably used, that thus fulfills a double function in the production of the capillary 21 and in its transport.

While the glass capillary is being heated and melted with the heating wire 72, the capillary 21 can be withdrawn with the capillary holder 23, so that the desired form of the capillary tip 22 results. Subsequently, the capillary 21 is deposited with the drive device 50, e.g., in a magazine 80, as is schematically illustrated in FIG. 11B. The magazine 80 comprises, e.g., a block 81 with holes into which capillaries 21 can be set, so that their capillary tips 22 remain free. The depositing takes place with a wipe-off apparatus or by exerting a pressure pulse in the capillary holder 23.

FIG. 12 shows further details of the use of a capillary for which a conical plug connection 24.1, 24.2 is provided. According to FIG. 12A a capillary 21 is used on whose ends the plug connections 24.1, 24.2 are fixed via screw connections 24.3, 24.4. The screw connections can be replaced by plug connections with at least one sealing ring (see FIG. 13B). The capillary 21 is inserted with plug connections 24.1, 24.2 into the clamping block 71 (see FIG. 11A) and drawn out. FIG. 12B shows the finished capillary 21 after drawing out.

FIG. 13 schematically shows a filling device 90 (FIG. 13A) provided for filling the capillary 21 of a patch-clamp pipette (FIG. 13B). The filling of the capillary of a patch-clamp pipette with an operating fluid (e.g., physiological solution) was previously associated with the risk of damaging the capillary. This problem occurs in particular when patch-clamp pipettes are used with capillaries that have different lengths as a function of the intended application. In order to avoid damage to the capillary the filling device 90 illustrated in FIG. 13A is preferably used.

The filling device 90 comprises a filling capillary 91 (e.g., a quartz capillary) that is fastened in a holding block 92. A connecting hose 93 is fastened on the holding block 92 that forms a liquid communication between the filling capillary 91 and a liquid transport mechanism 94 (e.g., injection device). A spring device 95 (e.g., a spiral spring) is provided between the liquid reservoir of the liquid transport device 94 and the holding block 92 on the outside of the connecting hose 93. The connecting hose 93 consists of a flexible material. The connection hose 93 and the spring device 95 can be elastically deformed. Forces that possibly unintentionally arise during the operation of the liquid transport device 94 can advantageously be taken up by an elastic deformation and thus an undesired transfer of force onto the capillary of the patch-clamp pipette is avoided.

FIG. 13B shows the combination of the filling device 90 with the capillary 21 of the patch-clamp pipette during the filling. The capillary 21 is set into a conical plug connection 24.5. The plug connection 24.5 (or: receiving device) comprises a conical plug with an axial bore on whose inner surface at least one sealing ring, but preferably two sealing rings 24.6 are arranged for holding the capillary 21. The axial bore comprises a funnel-shaped opening 24.7 with a stepped projection 24.8 that serves for the gentle introduction of the filling capillary 91 of the filling device 90 and for the formation of a stop for the capillary 21. The wall of the capillary 21 is advantageously covered by the projection 24.8 so that the filling capillary 91 is conducted by the funnel-shaped opening 24.7 directly into the inner space of the capillary 21.

An excessive stressing of the filling capillary 91 and of the capillary 21 is avoided by the possibility of an elastic deformation of the connecting hose 93 and of the spring mechanism 95. In particular, it is made possible that the introduced filling capillary 91 (as shown in FIG. 13B) can strike against the inner wall against the capillary 21 without forming excessive forces. This advantage is achieved on account of the deformability of the components 93, 95 irrespective of the length of the capillary 21, so that the filling device 90 is especially well suited for the filling of capillaries 21 of different lengths.

FIG. 14 shows by way of example further details of a capillary holder 23 of a patch-clamp pipette that can be used with advantage in the realization of the invention. The capillary holder 23 comprises a capillary electrode 23.1 that is preferably formed by a quartz capillary. The capillary electrode 23.1 is inserted in a carrier 23.2. The carrier 23.2 comprises a receptacle 23.3 on one end for the capillary (not shown) of the patch-clamp pipette and an electrolyte reservoir 23.5 on the opposite end with an electrode connection 23.6 (e.g., an Ag—AgCl pellet).

The receptacle 23.3 is arranged for directly receiving the capillary 21 or the plug connection 24.1, 24.2, 24.5 (see FIGS. 12, 13) and is preferably provided with at least one or two sealing rings 23.4. In order to receive the plug connection (e.g., 24.1, 24.2 or 24.5) the receptacle 23.3 preferably has a conical hollow form.

The capillary electrode 23.1 is preferably a quartz capillary filled with an electrolyte, for example, KCl, that empties into the electrolyte reservoir 23.5. Alternatively, an Ag—AgCl wire electrode can be provided.

The mechanisms shown in the FIGS. 11 to 14 can represent independent subject matter of the invention that can be realized with pipettes without the liquid holder device in accordance with the invention described above.

The features of the invention disclosed in the above description, the figures and the claims can be equally significant for realizing the invention in its different embodiments, either individually or in combination.

1. A liquid holder device arranged for forming a liquid environment on a capillary tip of a capillary of a patch-clamp pipette comprising:
a holding element, arranged for holding a liquid drop, and
at least one rod, arranged for positioning the holding element, on the capillary tip,
wherein the at least one rod is arranged in such a manner that in a state in which the liquid holder device is assembled with the patch-clamp pipette, the holding element can be separated from the capillary.

2. The liquid holder device according to claim 1, in which:
the holding element is arranged for holding the liquid drop under action of capillary forces, and
the holding element is pivotable with the at least one rod.

3. The liquid holder device according to claim 2, in which the at least one rod has a pivot hinge arranged for connecting the at least one rod to a capillary holder of the patch-clamp pipette.

4. The liquid holder device according to claim 3, in which the pivot hinge is equipped with a drive device for pivoting the at least one rod.

5. The liquid holder device according to claim 3, in which the pivot hinge is arranged for detachable connection of the at least one rod to the patch-clamp pipette.

6. The liquid holder device according to claim 5, in which the pivot hinge is arranged for clamping the at least one rod on the patch-clamp pipette.

7. The liquid holder device according to claim 2, in which the holding element is formed by a level plate, a profiled plate or a section of a hollow line cut in an axial direction.

8. The liquid holder device according to claim 7, in which the holding element is formed by a hollow cylinder cut in the axial direction.

9. The liquid holder device according to claim 2, in which the holding element is detachably fastened on the at least one rod.

10. The liquid holder device according to claim 1, in which:
the holding element is arranged for holding the liquid drop under action of gravitational force, and
the holding element is detachably fastened on the at least one rod.

11. The liquid holder device according to claim 10, in which the at least one rod is arranged for fixed fastening on the capillary or on a capillary holder of the patch-clamp pipette.

12. The liquid holder device according to claim 10, in which the holding element comprises a cup in whose wall an end of the at least one rod can be inserted.

13. The liquid holder device according to claim 12, in which an insertion opening in the wall and an inside of the cup are connected via a contact groove.

14. The liquid holder device according to claim 12, in which:
at least one conical insertion opening is provided in the wall of the cup, and
the at least one rod has a tapering end that fits into the at least one conical insertion opening.

15. The liquid holder device according to claim 10, which:
has two rods 2, arranged for positioning the holding element on the capillary tip, wherein
two insertion openings are provided in the wall of the cup that are arranged for receiving free ends of the rods.

16. The liquid holder device according to claim 1, in which the holding element is provided with an electrode.

17. The liquid holder device according to claim 16, in which the electrode is connected to the at least one rod or is formed by the at least one rod.

18. The liquid holder device according to claim 1, in which the holding element carries a hydrophilic or oleophilic coating.

19. The liquid holder device according to claim 1, in which the at least one rod is tubular.

20. A measuring instrument electrophysiological analyses, comprising:
a patch-clamp pipette comprising a capillary, a capillary tip arranged for receiving a biological cell, and a capillary holder arranged for receiving the capillary, and
a liquid holder device according to claim 1.

21. The measuring instrument according to claim 20, in which the at least one rod of the liquid holder device can be pivoted from a conditioning position in which the holding element contacts the capillary tip or is separated therefrom by a capillary gap into a release position in which the capillary tip is free.

22. The measuring instrument according to claim 20, in which the at least one rod is fixed on the capillary or the capillary holder and the holding element is detachably connected to the at least one rod.

23. The measuring instrument according to claim 20, further comprising a drive device arranged for moving and positioning the patch-clamp pipette and the liquid holder device.

24. The measuring instrument according to claim 20, in which the capillary is connected via a conical plug connection to the capillary holder.

25. The measuring instrument according to claim 20, further comprising a pipette drawing device arranged for production of the capillary.

26. A method for the electrophysiological analysis of a biological cell, using a liquid holder device and a measuring instrument according to claim 20, comprising the steps:
reception of the biological cell on the capillary tip of the patch-clamp pipette in a first surrounding medium,
transfer of the biological cell with the capillary tip into a second surrounding medium, wherein a liquid environment is formed with the holding element of the liquid holder device on the capillary tip of the patch-clamp pipette, wherein during or after the transfer of the biological cell a
measuring of an electrophysiological potential on the cell or of ionic currents through the cell membrane of the cell is provided.

27. The method according to claim 26, in which after the reception of the biological cell on the capillary tip of the patch-clamp pipette a pivoting of the at least one rod of the liquid holder device is provided in such a manner that the holding element rests on the capillary tip with the biological cell or is separated from it by a capillary gap.

28. The method according to claim 26, in which before the reception of the biological cell on the capillary tip of the patch-clamp pipette a fixing of the holding element on the at least one rod of the liquid holder device is provided in such a manner that the capillary tip extends into the holding element.

29. The method according to claim 26, further comprising the steps:
transferring the biological cell into at least one further surrounding medium, wherein during and/or after the transfer of the biological cell a further
measurement of the electrophysiological potential on the cell or of ionic currents through the cell membrane of the cell in the further surrounding medium is provided.

30. The method according to claim 26, in which the steps of reception, pivoting and transfer take place in an automated manner using an image recording device and a control device.

31. The method according to claim 26, in which solution is supplied via the at least one rod and the contact groove into the holding element or is removed from the latter.

32. The method according to claim 26, in which a production of the capillary of the patch-clamp pipette with a pipette drawing device is provided immediately before the reception of the biological cell.
